(19) United States
(12) Patent Application Publication
(10) Pub. No.: US 2026/0115158 A1
(43) Pub. Date: Apr. 30, 2026

(54) COMPOSITIONS FOR APPLICATION TO A SURFACE OF SKIN

(71) Applicant: KAO CORPORATION, Tokyo (JP)

(72) Inventors: Kathleen M. MCKEON, Cincinnati, OH (US); Monica L. OLTMANNS, Milford, OH (US)

(73) Assignee: KAO CORPORATION, Tokyo (JP)

(21) Appl. No.: 19/375,645

(22) Filed: Oct. 31, 2025

Related U.S. Application Data
(60) Provisional application No. 63/714,182, filed on Oct. 31, 2024.

(51) Int. Cl. A61K 31/192; A61K 9/00; A61K 31/719

(52) U.S. Cl. CPC A61K 31/192; A61K 9/0014; A61K 31/719

(57) ABSTRACT

A composition for application to a surface of skin includes a carrier, a plasticizer, a non-water solvent, salicylic acid, a gum and/or a polymer binder-film former, an occlusive agent, an emollient, and a pullulan. The composition may form a thin and durable protective film on the surface of skin after application. Methods of making and using the composition are further disclosed.

### CROSS REFERENCE TO RELATED APPLICATIONS

The present application claims priority to U.S. Application No. 63/714,182, filed on October 31, 2024, the contents of which are hereby incorporated in their entirety.

### FIELD OF TECHNOLOGY

Disclosed are compositions (for instance, film-forming compositions) for application to a surface of skin, as well as methods of making and using same.

### BACKGROUND

A person's appearance is typically of great importance. Skin conditions, including (but not limited to) acne, eczema, psoriasis, blemishes, and dry skin can cause anxiety and embarrassment to a person. Products exist to treat such skin conditions, often in the form of topical treatments such as liquids, creams, or gels.

While such products may successfully treat a skin condition, users may experience issues such as discomfort of the skin during treatment, chemical harshness from ingredients in a product, dryness of the skin after treatment, and even allergic reactions. A need exists for a product with improved properties for treating skin. The instant disclosure aims to address one or more of the aforementioned needs in the art.

### SUMMARY

Disclosed are compositions for application to a surface of skin, and methods of making and using the same. Such compositions may form a film on the surface of skin (i.e., film-forming compositions). The disclosed compositions may include a carrier, a plasticizer, a non-water solvent, salicylic acid, at least one of a gum and a polymer binder-film former, an occlusive agent, an emollient, and pullulan.

The disclosed compositions may be used to treat a surface of skin affected by conditions such as (but not limited to) acne, eczema, blemishes, and psoriasis.

### DETAILED DESCRIPTION

A composition for application to a surface of skin is presented. In certain aspects, the composition may form a film on the surface of skin (i.e., a film-forming composition). The film may be thin, durable, and protective to the surface of skin, and include an encapsulated salicylic acid. The composition may include a carrier, a plasticizer, a non-water solvent, salicylic acid, at least one of a gum and a polymer binder-film former, an occlusive agent, an emollient, and pullulan.

In certain aspects, the composition may be created by obtaining a carrier, forming a first dissolution, adding at least one of a gum and a polymer binder-film former to the first dissolution, then adding to the carrier, forming a second dissolution, then adding to the carrier, and then adding pullulan to the carrier. The first dissolution may include a plasticizer, a non-water solvent, and salicylic acid dissolved therein. The second dissolution may include an occlusive agent and an emollient.

For the purpose of this application, the term "dissolution" as used herein refers to a mixture or a solution of ingredients. A dissolution is not the composition (i.e., the fully formed composition), but rather an intermediate mixture or solution used to form the composition.

For the purpose of this application, the term "accumulate" as used herein refers to a collection (for instance, a mixture or a solution) of ingredients, including those that are not part of a dissolution. An accumulate may or may not include a dissolution. Ingredients that are part of an accumulate but not a dissolution may have functions that are different from those of the ingredients that are part of a dissolution. An accumulate is not the composition (i.e., the fully formed composition), but rather an intermediate mixture or solution that is used to form the composition.

For the purpose of this application, the term "substantially free of" may mean 1% or less of the specified component by weight of the composition. In certain versions, the term "substantially free of" may mean 0% of the specified component by weight of the composition. In certain versions, the term "substantially free of" may mean 0% of the specified component by weight of the composition.

For the purpose of this application, the term "wholly free of" may mean containing 0% of the specified component by weight of the composition.

### Carrier

In one aspect, the composition may include a carrier for subsuming the remaining ingredients of the composition. The carrier (water, for instance) is an ingredient of the composition into which all other ingredients of the composition are added and dissolved. The carrier may be present in an amount necessary to total a weight of the composition to 100% (QS). For instance, the carrier may be present in an amount ranging from 45% to 90%, or from 50% to 85%, or from 55% to 80%, or from 60% to 75%, or from 65% to 70% by weight of the composition. The carrier may be present in an amount ranging from 45%, or 50%, or 55%, or 60%, or 65% to 70%, or 75%, or 80%, or 85%, or 90% by weight of the composition. In one aspect, the carrier is water, and may be deionized, distilled, or purified water.

### First Dissolution

The composition may include a first dissolution. The first dissolution may include a plasticizer, a non-water solvent, and salicylic acid. Generally, the first dissolution may be formed by combining the ingredients of the first dissolution together with heating and/or mixing until a clear and homogeneous mixture or solution is formed.

The plasticizer (glycerin, for instance) is a multi-functional ingredient of the composition, primarily acting as a first solvent of the precursor dissolution (working with a non-water solvent to dissolve a salicylic acid and a gum and/or polymer binder-film former). Secondarily, the plasticizer acts as a humectant, a binder of the composition, and allows the composition to form a flexible film upon the surface of skin after application and drying. That is, the plasticizer allows the composition to be a film-forming composition. The plasticizer may be selected from: polyols, alkoxylated alcohols, carbohydrates, ethers, and esters. The plasticizer may be present in an amount ranging from 2.5% to 20%, or from 5% to 15%, or from 7.5% to 13% by weight of the composition. The amount may range from 2.5%, or 5%, or 7.5% to 13%, or 15%, or 20% by weight of the composition. Non-limiting examples of the plasticizer may include: glycerin, methyl gluceth-20, sorbitol, polyglycerin-10, erythritol, mannitol, maltitol, glucose, triethyl citrate, ethylcellulose (and) diisopropyl adipate, acetyl tributyl citrate, and combinations thereof.

The non-water solvent may be combined with the plasticizer to form a precursor dissolution, i.e., a precursor to the first dissolution. The non-water solvent (ethoxydiglycol, for instance) is an ingredient of the composition selected from ethers and alcohols that acts as a second solvent of the precursor dissolution (working with the plasticizer, or first solvent of the precursor dissolution, to dissolve a salicylic acid and a gum and/or polymer binder-film former). The non-water solvent may be present in an amount ranging from 0.5% to 4%, or from 1% to 3%, or from 1.5% to 2.5% by weight of the composition. The amount may range from 0.5%, or 1%, or 1.5% to 2.5%, or 3%, or 4% by weight of the composition. Non-limiting examples of the non-water solvent may include: ethoxydiglycol, benzyl alcohol, butylene glycol, dipropylene glycol, hexylene glycol, propylene glycol, alcohol (i.e., ethanol), dimethyl isosorbide, propanediol, isopentyldiol, and combinations thereof.

In certain aspects, a ratio of the plasticizer to the non-water solvent may range from 1:1 to 15:1, for instance, 1:1, 2:1, 2.5:1, 2.67:1 (8:3), 3:1, 3.33:1 (10:3), 4:1, 5:1, 5.5:1, 6:1, 10:1, or 13:1.

Salicylic acid may be dissolved into the precursor dissolution, thereby forming the first dissolution. Salicylic acid may function as an active for treating skin conditions such as acne, eczema, blemishes, and psoriasis, by breaking down outer layers of the surface of skin. Salicylic acid may be present in an amount of no greater than 20%, or less than 15%, or less than 13%, or less than 11%, or less than 10% by weight of the composition. For example, the amount of salicylic acid may range from 0.1% to 20%, or from 0.25% to 15%, or from 0.4% to 13%, or from 0.5% to 11%, or from 0.6% to 10%, or from 0.75% to 7.5%, or from 1% to 5%, or from 1.1% to 2.5%, or from 1.25% to 2% by weight of the composition. The amount may range from 0.1%, or 0.25%, or 0.4%, or 0.5%, or 0.6%, or 0.75%, or 1%, or 1.1%, or 1.25% to 2%, or 2.5%, or 5%, or 7.5%, or 10%, or 11%, or 13%, or 15%, or 20% by weight of the composition. Because salicylic acid is an acid, it may also act as a preservative by lowering a pH of the composition.

In certain embodiments, the amount of plasticizer may be greater than the amount of the salicylic acid. In such aspects, a ratio of the plasticizer to the salicylic acid may range from 2:1 to 25:1, or from 3:1 to 20:1, or from 5:1 to 15:1, or from 7:1 to 12:1. The ratio may range from 2:1, or 3:1, or 5:1, or 7:1 to 12:1, or 15:1, or 20:1, or 25:1. For instance, the ratio may be 3:1, 5:1, 6:1, 6.67:1 (20:3), 8:1, 8.33:1 (25:3), 10:1, 13:1, 20:1, or 22:1.

In certain embodiments, the amount of plasticizer and the amount of the salicylic acid, as well as a ratio of the plasticizer and the salicylic acid may vary from those stated in the preceding paragraph. For instance, the amount of plasticizer may be less than the amount of salicylic acid, and a ratio therebetween may range from 0.1:1 to 0.75:1, or from 0.2:1 to 0.5:1, or from 0.3:1 to 0.4:1. The ratio may range from 0.1:1, or 0.2:1, or 0.3:1 to 0.4:1, or 0.5:1, or 0.75:1.

The amount of non-water solvent may be greater than or equal to the amount of the salicylic acid. A ratio of the non-water solvent to the salicylic acid may range from 1:1 to 5:1, or from 1.5:1 to 4:1. The ratio may range from 1:1, or 1.5:1 to 4:1, or 5:1.

For instance, the ratio may be 1:1, 1.33:1 (4:3), or 2:1, or 2.5:1, or 4:1.

In one version, the first dissolution is substantially free of ethanol and surfactant. In another version the first dissolution is wholly free of ethanol and surfactant.

Non-limiting examples of other ingredients that may be part of the first dissolution may include: urea, zinc pyrithione, coal tar, diphenhydramine, chlorpheniramine, cetirizine, loratadine, fexofenadine, doxylamine, hydrocortisone, selenium sulfide, ketoconazole, lactic acid, niacinamide, camphor, calamine, retinal, adapalene, tretinoin, tazarotene, vitamin E (alpha-tocopherol), benzoyl peroxide, allantoin, azelaic acid, vitamin C (ascorbic acid), kojic acid, zinc oxide, undecylenic acid, benzoic acid, resorcinol, sodium borate, triacetin, zinc caprylate, propionic acid, benzalkonium chloride, colloidal oatmeal, benzocaine, witch hazel, tannic acid, zinc acetate, menthyl lactate, horse chestnut, aloe barbadensis, hyaluronic acid, sodium hyaluronate, vitamin B5 (panthenol), inulin, kaolin, and zinc pyrrolidone carboxylic acid.

### Gums and Polymer Binder-Film Formers

The composition may include at least one of a gum and a polymer binder-film former (i.e., a gum and/or a polymer binder-film former). The polymer binder-film former may be a polymer that may or may not be gum-derived. The gum and/or polymer binder-film former (Caesalpinia spinosa gum, for instance) is a multi-functional ingredient of the composition, primarily acting as a binder of the composition, allowing the composition to form a film upon the surface of skin, providing durability to a film formed by the composition, providing adhesion of the composition to the surface of skin, and increasing viscosity of the composition. The gums and/or polymer binder-film formers may be selected from: gums and their derivatives (including quaternary ammonium derivatives), hydrophilic colloids and their derivatives, alcohols, natural polymers, and synthetic polymers. The at least one of a gum and a polymer binder-film former (gum/and or polymer binder-film former) may be present in an amount ranging from 0.1% to 5%, or from 0.2% to 4%, or from 0.5% to 3%, or from 0.75% to 2.5%, or from 1% to 2% by weight of the composition. The amount may range from 0.1%, or 0.2%, or 0.5%, or 0.75%, or 1% to 2%, or 2.5%, or 3%, or 4%, or 5% by weight of the composition. Non-limiting examples of gums and/or polymer binder-film formers may include: Caesalpinia spinosa gum, hydroxypropylcellulose, xanthan gum, hydroxy acrylate/sodium acryloyldimethyl taurate copolymer & polyisobutene & PEG-7 trimethylolpropane coconut ether, cellulose gum, guar hydroxypropyltrimonium chloride, hydroxyethyl cellulose, hydroxypropyl methylcellulose, dextrin, sclerotium gum, sodium carboxymethyl starch, amylopectin, acaia senegal gum, Tamarindus indica seed gum, polyvinyl alcohol, PVP, polyester-11, polyester-37, VPNA copolymer, VP/dimethylaminoethylmethacrylate copolymer, alcohol (and) polyvinyl butyral, polyvinyl alcohol, and combinations thereof.

A ratio of salicylic acid to gum/and or polymer binder-film former may range from 0.3:1 to 2:1, or from 0.5:1 to 1.5:1. The ratio may range from 0.3:1 or 0.5:1 to 1.5:1 or 2:1. For instance, the ratio may be 0.33:1 (1:3), 0.5:1, 0.55:1 (1:1.8), 0.6:1, 0.67:1 (2:3), 0.75:1, 0.83:1 (2.5:3), 1:1, 1.11:1 (1:0.9), 1.33:1 (4:3), or 1.67:1 (5:3).

The at least one of a gum and a polymer binder-film former may be added to the first dissolution to form a subsequent dissolution.

### Second Dissolution

The composition may include a second dissolution. The second dissolution may include an occlusive agent and an emollient. The occlusive agent (caprylic/capric triglycerides, for instance) is a multi-functional ingredient of the composition that primarily prevents moisture loss from the surface of skin. Secondarily, the occlusive agent may provide skin conditioning, supplemental plasticizing (film formation of the composition upon the surface of skin), and slippage/spreadability of the composition across the surface of skin. The emollient (isodecyl neopentanoate, for instance) is a multi-functional ingredient of the composition that primarily provides skin conditioning and slippage of the composition across the surface of skin. Secondarily, the emollient may allow for film formation of the composition upon the surface of skin and act as a supplemental plasticizer.

The occlusive agent may be selected from: fats, oils, and esters. The occlusive agent may present in an amount ranging from 0.05% to 0.5%, or from 0.1% to 0.4%, or from 0.15% to 0.35% by weight of the composition. The amount may range from 0.05%, or 0.1%, or 0.15% to 0.35%, or 0.4%, or 0.5% by weight of the composition. Non-limiting examples of the occlusive agent may include: caprylic/capric triglycerides, triheptanoin, propylene glycol dicaprylate/dicaprate, propylene glycol dicaprate, dipentaerythrityl hexa Cs-Cg acid esters, and combinations thereof.

The emollient may be selected from esters. The emollient may be present in an amount ranging from 0.4% to 3.5%, or from 0.8% to 2.7%, or from 1% to 2.2%, or from 1.5% to 2% by weight of the composition. The amount may range from 0.4%, or 0.8%, or 1%, or 1.5% to 2%, 2.2%, or 2.7%, or 3.5% by weight of the composition. Non-limiting examples of the emollient may include: isodecyl neopentanoate, isononyl isononanoate, isopropyl palmitate, caprylic/capric glycerides, triheptanoin, caprylic/capric glycerides polyglyceryl-10 esters, and combinations thereof.

In certain aspects, some of these ingredients may function as both an occlusive agent and an emollient.

In certain aspects, the occlusive agent may be caprylic/capric triglycerides and the emollient may be isodecyl neopentanoate.

A ratio of the emollient to the occlusive agent may range from 3:1 to 10:1, for instance, 3:1, 4:1, 5:1, 5.67:1 (17:3), 6:1, 7:1, 8:1, 9:1, or 10:1.

The second dissolution, once formed, may be added to subsequent dissolution, or in certain aspects, added to the combined carrier and subsequent dissolution.

### Pullulan

Pullulan may be added to the compositions described herein after combination of the carrier, the subsequent dissolution, and the second dissolution. Pullulan is a polysaccharide polymer made up primarily of a-1, 6 linked maltotriose residues. The chemical structures of pullulan are variable depending upon carbon source, the microorganism that produces it (e.g., Aureobasidium pullulans) and fermentation conditions.

The composition may include a pullulan in an amount ranging from 2% to 18%, or from 4.5% to 13.5%, or from 6% to 12% by weight of the composition. The amount may range from 2%, or 4.5%, or 6% to 12%, or 13.5%, or 18% by weight of the composition. Pullulan may allow the composition to form a film on the surface of skin.

In certain versions, the pullulan may alternatively be a water-soluble polysaccharide, non-limiting examples of which may include pullulan derivatives such as: crosslinked pullulan, carboxy pullulan, carboxymethyl pullulan, sulfonated pullulan, sulfated pullulan, sulfopropyl pullulan, pullulan esters, diethylaminoethyl pullulan and acetylated pullulan, and combinations thereof. In versions including a water-soluble polysaccharide, the water-soluble polysaccharide may be present in similar amounts stated above for pullulan.

The amount of pullulan in the composition may be greater than the amount of the gum and/or polymer binder-film former. A ratio of pullulan to the gum/and or polymer binder-film former may range from greater than 1:1 to 25:1, or from 2:1 to 20:1, or from 3:1 to 15:1, or from 4:1 to 10:1. The ratio may range from greater than 1:1, or 2:1, or 3:1, or 4:1 to 10:1, or 15:1, or 20:1, or 25:1. For instance, the ratio may be 1.5:1, 2:1, 4:1, 4.25:1, 4.5:1, 5:1, 6:1, 9:1, 10:1, 12:1, 14:1, 16:1, or 24:1.

The amount of pullulan in the composition may be greater than the amount of the salicylic acid. A ratio of pullulan to the salicylic acid may range from 3:1 to 25:1, or from 5:1 to 20:1, or from 7.5:1 to 15:1. The ratio may range from 3:1, or 5:1, or 7.5:1 to 15:1, or 20:1, or 25:1. For instance, the ratio may be 3.6:1, 4.25:1, 5.4:1, 6:1, 7.2:1, 7.5:1, 9:1, 10.8:1, 12:1, 12.6:1, 14.4:1, 20:1, or 24:1.

A ratio of pullulan to the plasticizer may range from 0.3:1 to 3:1, or from 0.5:1 to 2.5:1, or from 0.6:1 to 2:1, or from 0.7:1 to 1.5:1, or from 0.8:1 to 1:1. The ratio may range from 0.3:1, or0.5:1, or0.6:1, or 0.7:1, or0.8:1 to 1:1, or 1.5:1, or 2:1, or 2.5:1, or 3:1. For instance, the ratio may be 0.36:1, 0.54:1, 0.72:1, or 0.75:1, or 0.85:1, or 0.9:1, or 1:1, or 1.08:1, or 1.09:1 (12:11), or 1.26:1, or 1.44:1, or 2.5:1.

A ratio of pullulan to the non-water solvent may range from 1:1 to 12:1, or from 2:1 to 10:1, or from 3:1 to 6:1. The ratio may range from 1:1, or 2:1, or 3:1 to 6:1, or 10:1, or 12:1. For instance, the ratio may be 1.8:1, 2:1, 2.5:1, 2.7:1, 3:1, 3.6:1, 4.5:1, 5.4:1, 6:1, 6.3:1, or 7.2:1, or 10:1, or 12:1.

Without intending to be bound by theory, it is believed that the salicylic acid becomes encapsulated within the precursor dissolution (formed from the plasticizer and the non-water solvent) while forming the first dissolution. This encapsulation is then entrapped within a gel network of pullulan and the gum and/or polymer binder-film former. When the composition is applied and adhered to, and dries upon, the surface of skin, a flexible film is formed. It is believed that the gel network of pullulan and the gum and/or polymer binder-film former encapsulating the salicylic acid allows for an effective treatment of a skin condition.

### Other Ingredients

The composition may also include other ingredients in addition to the ingredients listed above. Such ingredients may have functions such as (but not limited to): chelator, preservative (anti-microbial agent), pH adjuster, humectant, prebiotic, postbiotic, skin conditioning, antioxidant, stabilizer, and reducing tackiness of the composition.

In various versions, the composition may include a chelator for preventing adverse effects on stability or appearance of the composition by complexing or inactivating metal ions. The chelator may be selected from organic salts, alkyl-substituted amino acids, and amine oxides. The chelator may be present in an amount ranging from 0.035% to 0.3%, or from 0.075% to 0.25%, or from 0.1% to 0.2% by weight of the composition. The amount may range from 0.035%, to 0.075%, or 0.1% to 0.2%, or 0.25%, or 0.3% by weight of the composition. In certain versions, the chelator may be in aqueous form to facilitate inclusion into the composition. Non-limiting examples of the chelator may include: tetrasodium glutamate diacetate, disodium ethylenediaminetetraacetic acid ("EDTA"), sodium citrate, tetrasodium EDTA, sodium gluconate, and combinations thereof.

In various versions, the composition may include a preservative (for disrupting microbial cells in contact with the composition), such as: organic salts, quaternary ammonium compounds, alcohols, ethers, amides, amino acids, heterocyclic compounds, and halogen compounds. The preservative may be present in an amount ranging from 0.05% to 0.4%, or from 0.1% to 0.3%, or from 0.15% to 0.25% by weight of the composition. The amount may range from 0.05%, or 0.1%, or 0.15% to 0.25%, or 0.3%, or 0.4% by weight of the composition. In certain versions, the preservative may be in aqueous form to facilitate inclusion into the composition. Non-limiting examples of the preservative may include: sodium benzoate, benzalkonium chloride, potassium sorbate, phenoxyethanol, ethyl lauroyl arginate HCI, chlorhexidine digluconate, zinc pyrrolidone carboxylic acid, sodium dehydroacetate, and combinations thereof.

In various versions, the composition may include a pH-adjuster (for balancing a pH of the composition), such as inorganic bases. The pH-adjuster may be present in an amount ranging from 0.025% to 0.2%, or from 0.05% to 0.15%, or from 0.075% to 0.13% by weight of the composition. The amount may range from 0.025%, or 0.05%, or 0.075% to 0.13%, or 0.15%, or 0.2% by weight of the composition. In certain versions, the pH adjuster may be in aqueous form to facilitate inclusion into the composition. Non-limiting examples of the pH-adjuster may include: sodium hydroxide, ammonium hydroxide, potassium hydroxide, and combinations thereof.

In various versions, the composition may include additional ingredients that can have one, some, or all of the following non-limiting functions: humectant, prebiotic, postbiotic, skin conditioning, antioxidant, stabilizer, and reducing tackiness of the composition. Such additional ingredients may be selected from: carbohydrates, polyols, and biotechnological products. The additional ingredients may be present in an amount ranging from 0.25% to 2%, or from 0.5% to 1.5%, or from 0.75% to 1.3% by weight of the composition. The amount may range from 0.25%, or 0.5%, or 0.75% to 1.3%, or 1.5%, or 2% by weight of the composition. Non-limiting examples of the additional ingredients may include: inulin, xylitol, lactobacillus ferment, bacillus ferment filtrate extract, leuconostoc/radish root ferment filtrate, bifida ferment lysate, lactobacillus/rye flour ferment, bacillus ferment, pseudoalteromonas ferment extract, alpha-glucan oligosaccharide, and combinations thereof.

The composition may include combinations of the chelator, preservative, pH adjuster, and additional ingredients in certain versions.

### Other Possible Ingredients andor Excluded Ingredients

In certain versions, the composition may include ingredients, such as (but not limited to) ethanol, benzoyl peroxide, silicones, fatty-alcohols, colorants, and fragrances. In certain versions, the composition may be substantially free of ingredients such as (but not limited to): ethanol, benzoyl peroxide, silicones, fatty-alcohols, colorants, surfactants, and fragrances.

In certain versions, the composition may be wholly free of ethanol, benzoyl peroxide, silicones, fatty-alcohols, colorants, surfactants, and fragrances.

In one version, the composition is substantially free, or wholly free of starches, clays, and polyvinylpyrrolidone ("PVP"). This is not intended to be an exhaustive list.

The composition may be substantially free, or wholly free of any of the above ingredients in this section, and combinations thereof.

### Properties of the Composition

The composition may have the following properties. These are not meant to be exhaustive:

In certain versions, the composition may have a pH ranging from 2.5 to 10, or from 3 to 8.5, or from 4 to 7, or from 5 to 6. The pH may range from 2.5, or 3, or 4, or 5 to 6, or 7, or 8.5, or 10. In another version, the pH may range from 3 to 4. In other versions, the pH may be below 7. The pH of the composition may vary based on the intended application of the composition (for instance, for treatment of acne versus treatment of burns).

The composition may be in the form of a gel.

The composition may be sterile.

A film formed from the composition may be water-soluble.

A Maximum Force of Peeling is the greatest (i.e. maximum) force required to remove (i.e. peel) a film formed by the composition from a surface. A maximum force of peeling a film, formed by the composition, from a surface, may range from 0.25N to 0.45N, after 16 hours of drying on the surface, determined using the method described below. Such a surface may be of artificial leather. Such a film may have a weight before drying ranging from 0.18g to 0.2g, and a weight after drying ranging from 0.04g to 0.08g. The film may have a basis weight ranging from 45 g/m2 to 55 g/m2, or from 48 g/m2 to 52 g/m2, or from 49 g/m2 to 51 g/m2. The area of application over the surface may range from 1000 mm2 to 1500 mm2, or from 1100 mm2 to 1400 mm2, or from 1150 mm2 to 1300 mm2, for instance 1200 mm2.

The method ("Maximum Peeling Force Method") for determining the maximum force required to peel a film, formed by the composition, from a surface is described herein. A sample of artificial leather (for instance, ldeatex Japan Co. Product # PBZ13001, Lot# D94-1091) is obtained. The sample may be, for instance, 95 mm by 150 mm in area. The composition may be applied in an amount ranging from 0.17 to 0.2g centrally to the sample and gently spread (for instance, over an area of 60 mm by 20 mm) and allowed to dry for 16 hours to form a film upon the sample. Double-sided sticky tape is applied to a back side, along a width, of the sample. The sample is adhered to a testing device surface (IMADA® 180° Peeling Tester IPTS, Model IPTS-1 with load cell serial# 426467) with a length of the sample parallel to the testing device stage. A film grip is moved toward a furthest edge (i.e., an edge furthest from the film grip) of the sample. The film grip may, for instance, have a screw mechanism on top that allows for clamping onto the film. Using a pair of tweezers, a portion of the film is manually peeled up from the sample (for instance, 1 cm along the length of the film) and the film is further peeled using the tweezers (for instance 2 mm to 3 mm along the length of the film), then clamped using the film grip attached to the testing device. The testing device is zeroed to tare the attachment force, then the method is started by pressing the "start" button. The testing device is then allowed to operate, peeling the film off from the sample. The testing device is stopped by pressing the "stop" button, and the maximum force of peeling is recorded. The process may be repeated, for instance, done for a total of three samples, and an average maximum force with standard deviation can be obtained via external software. Standard room operating temperature may be from 20°C to 25°C (for instance, 22.2°C) and 50% humidity. It is possible that the film may tear during testing, and thus the entire film may not be removed from the sample. The portion of the film that is removed is typically fully removed from the sample.

### Exemplary Formulations

The following are exemplary, non-limiting formulations of the composition as disclosed herein. Percentages shown are weight percentages of the total composition, unless otherwise stated:

Example 1: purified water (75.55%), tetrasodium glutamate diacetate (0.15%), sodium benzoate (0.2%), sodium hydroxide (0.1%), glycerin (8%), ethoxydiglycol (3%), salicylic acid (1%), Caesalpinia spinosa gum (3%), caprylic/capric triglycerides (0.5%), triheptanoin (1.5%), inulin (1%), pullulan (6%).

Example 2: purified water (64.55%), tetrasodium glutamate diacetate (0.15%), sodium benzoate (0.2%), sodium hydroxide (0.1%), glycerin (6%), ethoxydiglycol (6%), salicylic acid (2%), Caesalpinia spinosa gum (3%), dipentaerythrityl hexa Cs-9 acid esters (0.25%), caprylic/capric glycerides (1.75%), inulin (1%), pullulan (15%).

Example 3: purified water (72.05%), tetrasodium EDTA (0.15%), sodium benzoate (0.2%), sodium hydroxide (0.1%), glycerin (10%), ethoxydiglycol (2%), salicylic acid (1.5%), hydroxypropylcellulose (2%), caprylic/capric triglycerides (0.25%), isononyl isononanoate (1.75%), lactobacillus ferment (1%), pullulan (9%).

Example 4: purified water (71.05%), tetrasodium glutamate diacetate (0.15%), phenoxyethanol (0.5%), sodium hydroxide (0.1%), sorbitol (10%), dipropylene glycol (3%), salicylic acid (1.2%), hydroxy acrylate/sodium acryloyldimethyl taurate copolymer & polyisobutene & PEG-7 trimethylolpropane coconut ether (2%), propylene glycol dicaprate (0.25%), isodecyl neopentanoate (1.75%), inulin (1%), pullulan (9%).

Example 5: purified water (70.55%), disodium EDTA (0.15%), sodium benzoate (0.2%), sodium hydroxide (0.1%), polyglycerin-10 (10%), triethyl citrate (1%), ethoxydiglycol (2%), salicylic acid (0.5%), xanthan gum (1%), caprylic/capric triglycerides (0.25%), caprylic/capric glycerides polyglyceryl-10 esters (1.75%), inulin (0.5%), pullulan (12%).

Example 6: purified water (68.55%), tetrasodium glutamate diacetate (0.15%), sodium benzoate (0.2%), sodium hydroxide (0.1%), glycerin (10%), ethoxydiglycol (2%), salicylic acid (1%), Caesalpinia spinosa gum (0.6%), caprylic/capric triglycerides (0.25%), isodecyl neopentanoate (1.75%), inulin (1%), pullulan (14.4%).

Example 7: purified water (70.05%), tetrasodium glutamate diacetate (0.15%), sodium benzoate (0.2%), sodium hydroxide (0.1%), glycerin (10%), ethoxydiglycol (2%), salicylic acid (1%), Caesalpinia spinosa gum (0.9%), caprylic/capric triglycerides (0.25%), isodecyl neopentanoate (1.75%), inulin (1%), pullulan (12.6%).

Example 8: purified water (71.55%), tetrasodium glutamate diacetate (0.15%), sodium benzoate (0.2%), sodium hydroxide (0.1%), glycerin (10%), ethoxydiglycol (2%), salicylic acid (1%), Caesalpinia spinosa gum (1.2%), caprylic/capric triglycerides (0.25%), isodecyl neopentanoate (1.75%), inulin (1%), pullulan (10.8%).

Example 9: purified water (74.55%), tetrasodium glutamate diacetate (0.15%), sodium benzoate (0.2%), sodium hydroxide (0.1%), glycerin (10%), ethoxydiglycol (2%), salicylic acid (1%), Caesalpinia spinosa gum (1.8%), caprylic/capric triglycerides (0.25%), isodecyl neopentanoate (1.75%), inulin (1%), pullulan (7.2%).

Example 10: purified water (76.05%), tetrasodium glutamate diacetate (0.15%), sodium benzoate (0.2%), sodium hydroxide (0.1%), glycerin (10%), ethoxydiglycol (2%), salicylic acid (1%), Caesalpinia spinosa gum (2.1%), caprylic/capric triglycerides (0.25%), isodecyl neopentanoate (1.75%), inulin (1%), pullulan (5.4%).

Example 11: purified water (77.55%), tetrasodium glutamate diacetate (0.15%), sodium benzoate (0.2%), sodium hydroxide (0.1%), glycerin (10%), ethoxydiglycol (2%), salicylic acid (1%), Caesalpinia spinosa gum (2.4%), caprylic/capric triglycerides (0.25%), isodecyl neopentanoate (1.75%), inulin (1%), pullulan (3.6%).

Example 12: purified water (72.55%), tetrasodium glutamate diacetate (0.15%), sodium benzoate (0.2%), sodium hydroxide (0.1%), glycerin (9%), ethoxydiglycol (3%), salicylic acid (1.5%), Caesalpinia spinosa gum (1.5%), caprylic/capric triglycerides (0.5%), isodecyl neopentanoate (1.5%), inulin (1%), pullulan (9%).

Example 13: purified water (70.05%), tetrasodium glutamate diacetate (0.15%), sodium benzoate (0.2%), sodium hydroxide (0.1%), glycerin (10%), ethoxydiglycol (4%), salicylic acid (2%), Caesalpinia spinosa gum (2%), caprylic/capric triglycerides (0.25%), isodecyl neopentanoate (1.75%), inulin (1%), pullulan (8.5%).

Example 14: purified water (73.05%), tetrasodium glutamate diacetate (0.15%), sodium benzoate (0.2%), sodium hydroxide (0.1%), glycerin (10%), ethoxydiglycol (2%), salicylic acid (1%), Caesalpinia spinosa gum (1.5%), caprylic/capric triglycerides (0.25%), isodecyl neopentanoate (1.75%), inulin (1%), pullulan (9%).

Example 15: purified water (72%), tetrasodium glutamate diacetate (2%), sodium benzoate (0.2%), sodium hydroxide (0.1%), glycerin (10%), ethoxydiglycol (1%), salicylic acid (0.5%), Caesalpinia spinosa gum (1%), caprylic/capric triglycerides (0.25%), isodecyl neopentanoate (1.75%), inulin (1.2%), pullulan (10%).

Example 16: purified water (68.3%), tetrasodium glutamate diacetate (0.15%), sodium benzoate (0.2%), sodium hydroxide (0.1%), glycerin (13%), ethoxydiglycol (1%), salicylic acid (1%), Caesalpinia spinosa gum (0.75%), caprylic/capric triglycerides (0.3%), isodecyl neopentanoate (1.7%), inulin (1.5%), pullulan (12%).

Example 17: Purified water (QS), tetrasodium glutamate diacetate (0.15%), phenoxyethanol (0.2%), sodium hydroxide (0.1%), glycerin (10%), ethoxydiglycol (2%), salicylic acid (1%), Caesalpinia spinosa gum (1.5%), caprylic/capric triglycerides (0.25%), isodecyl neopentanoate (1.75%), pullulan (9%), inulin (1%).

### Methods of Making

The following are exemplary, non-limiting methods of making the composition as disclosed herein:

### Method 1

Add carrier, chelator, preservative, and pH-adjuster one at a time, mixing and heating to 70°C-80°C into a main mixing vessel to form a first accumulate.

In a first separate mixing vessel, combine plasticizer, non-water solvent, and salicylic acid, mixing and heating to 70°C-80°C, forming a first dissolution of the composition. Heat and mix until the first dissolution is clear and homogeneous.

Cool the first dissolution to 25°C-35°C, continue mixing, and add at least one of gum and polymer binder-film former (i.e., gum and/or polymer binder-film former) to the first dissolution to form a subsequent dissolution. When there are no more clumps, add the subsequent dissolution to the first accumulate, forming a second accumulate.

Continue mixing for at least 5 minutes, then begin cooling the second accumulate to 35°C- 450C while mixing for at least 30 minutes and ensuring no clumps.

In a second separate mixing vessel, combine and mix occlusive agent and emollient to form a second dissolution of the composition. Then add the second dissolution to the second accumulate, forming a third accumulate.

Slowly add remaining additional other ingredients (for instance, chelator, preservative, pH-adjuster, humectant) into the third accumulate, ensuring homogeneity and no clumps, to form a fourth accumulate.

Slowly add pullulan into the fourth accumulate and continue mixing for at least 30 minutes, ensuring homogeneity and no clumps, to form a final mixture (the composition).

### Method 2

Add carrier, chelator, preservative and pH-adjuster one at a time, mixing and heating to 70°C-80°C into a main mixing vessel to form a first accumulate.

In a first separate mixing vessel, combine plasticizer, non-water solvent, and salicylic acid, mixing and heating to 70°C-80°C, forming a first dissolution of the composition. Heat and mix until the first dissolution is clear and homogenous.

Cool the first dissolution to 25°C-35°C, continue mixing, and add at least one of gum and polymer binder-film former (i.e., gum and/or polymer binder-film former) to the first dissolution to form a subsequent dissolution. Once fully dispersed, homogenize the subsequent dissolution. After homogenization, add the subsequent dissolution to the first accumulate, forming a second accumulate. Continue mixing for at least 5 minutes, then begin cooling the second accumulate to 35°C-45°C while mixing for at least 30 minutes and ensuring no clumps.

In a second separate mixing vessel, combine and mix occlusive agent and emollient to form a second dissolution of the composition. Then add the second dissolution to the second accumulate, forming a third accumulate.

Slowly add remaining additional other ingredients (for instance, chelator, preservative, pH-adjuster, humectant) into the third accumulate, ensuring homogeneity and no clumps, to form a fourth accumulate.

Slowly add pullulan into the fourth accumulate and continue mixing for at least 30 minutes, ensuring homogeneity and no clumps, to form a final mixture (the composition).

### Method 3

Add carrier, chelator, preservative and pH-adjuster one at a time, mixing and heating to 70°C-80°C into a main mixing vessel to form a first accumulate.

In a first separate mixing vessel, combine plasticizer, non-water solvent, and salicylic acid, mixing and heating to 70°C-80°C, forming a first dissolution of the composition. Heat and mix until the first dissolution is clear and homogenous.

Cool the first dissolution to 25°C-35°C, continue mixing, and add at least one of gum and polymer binder-film former (i.e., gum and/or polymer binder-film former) to the first dissolution to form a subsequent dissolution. When there are no more clumps, add the subsequent dissolution to the first accumulate, forming a second accumulate. Continue mixing for at least 5 minutes, then begin cooling the second accumulate to 35°C- 450C while mixing for at least 30 minutes and ensuring no clumps. Homogenize the second accumulate. After homogenizing, return to mixing and proceed to the next step.

In a second separate mixing vessel, combine and mix occlusive agent and emollient to form a second dissolution of the composition. Then add the second dissolution to the second accumulate, forming a third accumulate.

Slowly add remaining additional other ingredients (for instance, chelator, preservative, pH-adjuster, humectant) into the third accumulate, ensuring homogeneity and no clumps, to form a fourth accumulate.

Slowly add pullulan into the fourth accumulate and continue mixing for at least 30 minutes, ensuring homogeneity and no clumps, to form a final mixture (the composition).

### Method 4

Add carrier, chelator, preservative, and pH-adjuster one at a time, mixing and heating to 70°C-80°C into a main mixing vessel to form a first accumulate.

In a first separate mixing vessel, combine plasticizer, non-water solvent, and salicylic acid, mixing and heating to 70°C-80°C, forming a first dissolution of the composition. Heat and mix until the first dissolution is clear and homogenous.

Cool the first dissolution to 25°C-35°C, continue mixing, and add at least one of gum and polymer binder-film former (i.e., gum and/or polymer binder-film former) to the first dissolution to form a subsequent dissolution. When there are no more clumps, add the subsequent dissolution to the first accumulate, forming a second accumulate.

Continue mixing for at least 5 minutes, then begin cooling the second accumulate to 35°C- 450C while mixing for at least 30 minutes and ensuring no clumps.

In a second separate mixing vessel, combine and mix occlusive agent and emollient to form a second dissolution of the composition. Then add the second dissolution to the second accumulate, forming a third accumulate.

Slowly add remaining additional other ingredients (for instance, chelator, preservative, pH-adjuster, humectant) into the third accumulate, ensuring homogeneity and no clumps, to form a fourth accumulate.

Slowly add pullulan into the fourth accumulate and continue mixing for at least 30 minutes, ensuring homogeneity and no clumps, to form a final mixture (the composition). Homogenize the final mixture.

### Method 5

Add carrier, chelator, preservative, and pH-adjuster one at a time, mixing into a main mixing vessel to form a first accumulate.

In a first separate mixing vessel, combine plasticizer, non-water solvent, and salicylic acid, mixing and heating to 70°C-80°C, forming a first dissolution of the composition. Heat and mix until the first dissolution is clear and homogenous.

Cool the first dissolution to 25°C-35°C, continue mixing, and add at least one of gum and polymer binder-film former (i.e., gum and/or polymer binder-film former) to the first dissolution to form a subsequent dissolution. When there are no more clumps, add the subsequent dissolution to the first accumulate, forming a second accumulate. Continue mixing for at least 30 minutes and ensuring no clumps.

In a second separate mixing vessel, combine and mix occlusive agent and emollient to form a second dissolution of the composition. Then add the second dissolution to the second accumulate, forming a third accumulate.

Slowly add remaining additional other ingredients (for instance, chelator, preservative, pH-adjuster, humectant) into the third accumulate, ensuring homogeneity and no clumps, to form a fourth accumulate.

Slowly add pullulan into the fourth accumulate and continue mixing for at least 30 minutes, ensuring homogeneity and no clumps, to form a final mixture (the composition).

### Method 6

Add carrier, chelator, preservative, and pH-adjuster one at a time, mixing into a main mixing vessel to form a first accumulate.

In a first separate mixing vessel, combine plasticizer, non-water solvent, and salicylic acid, mixing and heating to 45°C-55°C, forming a first dissolution of the composition. Heat and mix until the first dissolution is clear and homogenous.

Cool the first dissolution to 25°C-35°C, continue mixing, and add at least one of gum and polymer binder-film former (i.e., gum and/or polymer binder-film former) to the first dissolution to form a subsequent dissolution. When there are no more clumps, add the subsequent dissolution to the first accumulate, forming a second accumulate. Continue mixing for at least 30 minutes and ensuring no clumps.

In a second separate mixing vessel, combine and mix occlusive agent and emollient to form a second dissolution of the composition. Then add the second dissolution to the second accumulate, forming a third accumulate.

Slowly add remaining additional other ingredients (for instance, chelator, preservative, pH-adjuster, humectant) into the third accumulate, ensuring homogeneity and no clumps, to form a fourth accumulate.

Slowly add pullulan into the fourth accumulate and continue mixing for at least 30 minutes, ensuring homogeneity and no clumps, to form a final mixture (the composition).

### Method 7

Add carrier, chelator, preservative, and pH-adjuster one at a time, mixing into a main mixing vessel to form a first accumulate.

In a first separate mixing vessel, combine plasticizer, non-water solvent, and salicylic acid, mixing to form a first dissolution of the composition. Heat and mix until the first dissolution is clear and homogenous. Add at least one of gum and polymer binder-film former (i.e., gum and/or polymer binder-film former) to the first dissolution to form a subsequent dissolution. When there are no more clumps, add the subsequent dissolution to the first accumulate, forming a second accumulate. Continue mixing for at least 30 minutes and ensuring no clumps.

In a second separate mixing vessel, combine and mix occlusive agent and emollient to form a second dissolution of the composition. Then add the second dissolution to the second accumulate, forming a third accumulate.

Slowly add remaining additional other ingredients (for instance, chelator, preservative, pH-adjuster, humectant) into the third accumulate, ensuring homogeneity and no clumps, to form a fourth accumulate.

Slowly add pullulan into the fourth accumulate and continue mixing for at least 30 minutes, ensuring homogeneity and no clumps, to form a final mixture (the composition).

### Method of Use

The following are general, non-limiting methods of use of the composition as disclosed herein.

In one aspect, a method of applying the composition to a surface of skin is herein disclosed. A user dispenses an amount of the composition (for instance, a dime-sized amount, as a disc or drop ranging from 15mm to 25 mm in diameter, and typically ranging from 0.05g to 0.15g per site of acne) from a container (for instance, via squeezing from a tube, or dipping an applicator into a bottle) onto a finger or an applicator (for instance, a brush, a cotton swab, a spatula, or a foam pad) that may be provided with the container.

The composition is then painted onto the surface of skin (previously cleaned and dried) in a thick, even layer (for instance, over a site of acne). The composition is allowed to fully dry into a film-like patch. Drying typically occurs for 10 to 15 minutes. The composition, now dried, is then allowed to remain in place for a minimum of at least 2 hours, for instance, at least 2 hours, at least 4 hours, or at least 6 hours.

In one aspect, a method of removing the composition from the surface of skin is herein disclosed. The user gently rubs an outer edge of the film-like patch, causing it to slightly raise. Using a fingernail, the user lifts the film-like patch (and thus, the composition) off from the surface of skin, slowly peeling off in one piece.

Alternatively, if the film-like patch is fragmented during removal, or the outer edge is not located, the user may wash off with water and gentle rubbing.

The above methods can be repeated until the site of acne is cleared. If eruption of the site of acne occurs, a user should discontinue applying the composition to avoid disruption of scab formation.

In some instances (e.g., if the surface of skin is oily), a user may apply a toner immediately prior to application of the composition, which may provide better adhesion of the composition to the surface of skin.

All percentages and ratios are calculated by weight unless otherwise indicated. All percentages and ratios are calculated based on the total weight of the composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Percent ranges of amounts, ratios, and other numerical values, as disclosed herein, are understood to include the recited value +/-1% unless otherwise specified. As used herein, articles such as "a" and "an" when used in a claim, are understood to mean one or more of what is claimed or described.

As used herein, an amount of an ingredient is the percent by weight that the ingredient comprises within the total composition. The total composition comprises an amount of 100%.

As used herein, the terms "comprising," "comprises," "include," "includes," and "including" are meant to be non-limiting. The terms "consisting of" or "consisting essentially of" are meant to be limiting, i.e., excluding any components or ingredients that are not specifically listed except when they are present as impurities.

All references, including patent applications, patent publications, and non-patent literature, that are referred to in the present specification are incorporated by reference herein, unless it is expressly indicated that they are not incorporated by reference herein. Having shown and described various aspects of the present invention, further adaptations of the compositions and methods described herein may be accomplished by appropriate modifications by one of ordinary skill in the art without departing from the scope of the present invention. Several of such potential modifications have been mentioned, and others will be apparent to those skilled in the art. For instance, the examples, materials, ratios, steps, and the like discussed above are illustrative and are not required. Accordingly, the scope of the present invention should be considered in terms of any claims that may be presented and is understood not to be limited to the details of composition and operation described in the specification.

1. A composition, for application to a surface of skin, comprising:
a carrier;
a plasticizer,
a non-water solvent;
salicylic acid;
at least one of a gum and a polymer binder-film former;
an occlusive agent;
an emollient; and
pullulan,
wherein pullulan is in a greater amount than the at least one of a gum and a polymer binder-film former, and a weight ratio therein ranges from greater than 1:1 to 25:1.

2. The composition of claim 1, wherein an amount of the plasticizer is greater than an amount of salicylic acid, and
an amount of the non-water solvent is greater than or equal to the amount of salicylic acid.

3. The composition of claim 1, wherein a weight ratio of pullulan to salicylic acid ranges from 3:1 to 25:1.

4. The composition of claim 1, wherein the weight ratio of pullulan and the at least one of the gum and the polymer binder-film former ranges from 2:1 to 20:1.

5. The composition of claim 1, wherein a weight ratio of the emollient and the occlusive agent ranges from 3-1 to 10:1.

6. The composition of claim 1, exhibiting a maximum peeling force ranging from 0.25 N to 0.45 N, according to the Maximum Peeling Force Method.

7. A composition, for application to a surface of skin, comprising:
a carrier;
a plasticizer;
a non-water solvent;
salicylic acid;
at least one of a gum and a polymer binder-film former;
an occlusive agent;
an emollient; and
pullulan,
wherein pullulan is in a greater amount than salicylic acid,
pullulan is in a greater amount than the at least one of a gum and a polymer binder-film former and a weight ratio therein ranges from greater than 1:1 to 25:1, and
wherein an amount of the plasticizer is greater than an amount of salicylic acid, and an amount of the non-water solvent is greater than or equal to the amount of salicylic acid.

8. The composition of claim 7, wherein a weight ratio of the plasticizer to the salicylic acid ranges from 2:1 to 25:1.

9. The composition of claim 7, wherein a weight ratio of the non-water solvent to salicylic acid ranges from greater than 1:1 to 5:1.

10. The composition of claim 7, wherein a weight ratio of pullulan to the plasticizer ranges from 0.3:1 to 3:1.

11. The composition of claim 7, wherein a weight ratio of pullulan to the non-water solvent ranges from 1:1 to 12:1.

12. The composition of claim 7, wherein the weight ratio of pullulan to the at least one of the gum and the polymer binder-film former ranges from 2:1 to 20:1.

13. The composition of claim 7, wherein a weight ratio of the plasticizer to the non-water solvent ranges from 1:1 to 15:1.

14. The composition of claim 7, wherein a weight ratio of pullulan to salicylic acid ranges from 3:1 to 25:1.

15. The composition of claim 14, wherein the weight ratio of pullulan to salicylic acid ranges from 5:1 to 20:1.

16. A method of making a composition for application to a surface of skin, the method comprising:
adding a carrier into a main mixing vessel;
combining, in a first separate mixing vessel, a plasticizer, a non-water solvent, and salicylic acid to form a first dissolution of the composition;
heating and mixing the first dissolution until clear and homogeneous;
adding at least one of a gum and a polymer binder-film former to the first dissolution to form a subsequent dissolution;
adding the subsequent dissolution to the main mixing vessel;
mixing the carrier and the subsequent dissolution in the main mixing vessel for at least 30 minutes;
combining, in a second separate mixing vessel, an occlusive agent and an emollient to form a second dissolution of the composition;
adding the second dissolution to the main mixing vessel;
adding pullulan to the main mixing vessel to form the composition; and
mixing the composition for at least 30 minutes, ensuring homogeneity.

17. The method of claim 16, further comprising:
adding a chelator, a preservative, and a pH-adjuster one at a time, mixing into the main mixing vessel, prior to adding the subsequent dissolution of the composition; and
adding additional ingredients to the main mixing vessel, after adding the second dissolution of the composition and prior to adding pullulan.

18. The method of claim 17, wherein the carrier, the chelator, the preservative, and the pH-adjuster are mixed and heated to 70 °C - 80 °C.

19. The method of claim 16, wherein the plasticizer, the non-water solvent, and salicylic acid are mixed and heated to 70 °C - 80 °C.

20. The method of claim 16, wherein the plasticizer, the non-water solvent, and salicylic acid are mixed and heated to 45 °C - 55 °C.
